(19) United States
(12) Patent Application Publication
(10) Pub. No.: US 2015/0299381 A1
(43) Pub. Date: Oct. 22, 2015

(54) POLYMERIC PRECURSORS FOR PRODUCING GRAPHENE NANORIBBONS AND SUITABLE OLIGOPHENYLENE MONOMERS FOR PREPARING THEM

(71) Applicants: BASF SE, Ludwigshafen (DE); MAX-PLANCK-GESELLSCHAFT ZUR FÖRDERUNG DER WISSENSCHAFTEN E.V., München (DE)

(72) Inventors: Klaus MUELLEN, Mainz (DE); Xinliang FENG, Mainz (DE); Jinming CAI, Zuerich (CH); Pascal RUFFIEUX, Plasselb (CH); Roman FASEL, Zuerich (CH); Akimitsu NARITA, Mainz (DE)

(73) Assignees: BASF SE, Ludwigshafen (DE); Max-Planck-Gesellschaft zur Foerderung der Wissenschaften e.V., Muenchen (DE)

(21) Appl. No.: 14/443,250

(22) PCT Filed: Nov. 12, 2013
(86) PCT No.: PCT/IB13/60075

§ 371 Date: May 15, 2015

Related U.S. Application Data
(60) Provisional application No. 61/727,157, filed on Nov. 16, 2012.

(51) Int. Cl. C08G 61/12; C01B 31/04; C07C 25/18

(52) U.S. Cl. CPC C08G 61/121; C07C 25/18; C01B 31/0438; C08G 2261/312; C01B 2204/06; C01B 2204/22; H01L 51/102

(57) ABSTRACT

The invention relates to oligophenylene monomers of general formula I, wherein R¹ is H, halogene, —OH, —NH₂, —CN, —NO₂, or a linear or branched, saturated or un-saturated C₁-C₄₀ hydrocarbon residue, which can be substituted 1- to 5-fold with halogene (F, Cl, Br, I), —OH, —NH₂, —CN and/or —NO₂, and wherein one or more CH₂-groups can be replaced by —O—, —S—, —C(O)O—, —O—C(O)—, —C(O)—, —NH— or —NR³—, wherein R³ is an optionally substituted C₁-C₄₀ hydrocarbon residue, or an optionally substituted aryl, al-kylaryl, alkoxyaryl, alkanoyl or aroyl residue; R^{2a} and R^{2b} are H, or optionally one or more of the pairs of adjacent R^{2a}/R^{2b} is joined to form a single bond in a six-membered carbocycle; m is an integer of from 0 to 3; n is 0 or 1; and X is halogene or trifluoromethylsulfonate, and Y is II; or X is II, and Y is halogene or trifluoromethylsulfonate. The invention further relates to polymeric precursors as well as methods for preparing graphene nanoribbons from the oligophenylene monomers and the polymeric precursors.

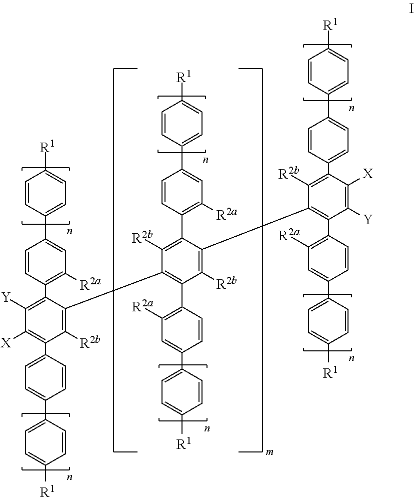

The present invention concerns oligophenylene monomers for the synthesis of polymeric precursors for the preparation of graphene nanoribbons, the polymeric precursors, and methods for preparing them, as well as methods for preparing the graphene nanoribbons from the polymeric precursors and the monomers.

Graphene, an atomically thin layer from graphite, has received considerable interest in physics, material science and chemistry since the recent discovery of its appealing electronic properties. These involve superior charge carrier mobility and the quantum Hall effect. Moreover, its chemical robustness and material strength make graphene an ideal candidate for applications ranging from transparent conductive electrodes to devices for charge and energy storage.

Graphene nanoribbons (GNRs) are linear structures that are derived from the parent graphene lattice. Their characteristic feature is high shape-anisotropy due to the increased ratio of length over width. Currently, their usage in yet smaller, flatter and faster carbon-based devices and integrated circuits is being widely discussed in material science. In contrast to graphene, armchair-type GNRs exhibit a band gap that can be adjusted by their width. Their length becomes relevant when GNRs are to be used in devices such as field-effect transistors (FETs) for which a minimum channel width has to be bridged. The same holds for the potential replacement of copper or gold in nanoscale conducting pathways. At the same time the edge structure of the GNRs will have a strong impact. Computational simulations and experimental results on smaller nanographenes suggest that GNRs exhibiting nonbonding TT-electron states at zigzag edges could be used as active component in spintronic devices.

The reason why there are so few chemically defined GNRs is the considerable complexity that governs design, chemical preparation and processing of these structures. In the recent past, only few synthetic attempts have been published addressing the fabrication of GNRs of defined geometry, width, length, edge structure and heteroatom-content. Based on the reaction environment the studies on the synthetic bottom-up fabrication of GNRs can be further divided into solution- and surface-based routes.

For solution-based approaches using oligophenylene precursors a polymer is typically prepared in a first step which is subsequently converted into the graphitic structure by Scholl-type oxidative cyclodehydrogenation. However, the design of the parent monomer must be carefully adjusted in order to guarantee for a suitable arrangement of the aromatic units upon the chemistry-assisted graphitization into the final GNR structure.

J. Wu, L. Gherghel, D. Watson, J. Li, Z. Wang, C. D. Simpson, U. Kolb, and K. Müllen, Macromolecules 2003, 36, 7082-7089 report the synthesis of graphitic nanoribbons obtained by intramolecular oxidative cyclodehydrogenation of soluble branched polyphenylenes, which were prepared by repetitive Diels-Alder cycloaddition of 1,4-bis(2,4,5-triphenylcyclopentadienone-3-yl)benzene and diethynylterphenyl. The obtained graphene ribbons are not linear but rather contain statistically distributed “kinks” due to the structural design of the polyphenylene precursor.

X. Yang., X. Dou, A. Rouhanipour, L. Zhi, H. J. Räder, and K. Müllen, JACS Communications, Published on Web 03/07/2008, report the synthesis of two-dimensional graphene nanoribbons. Suzuki-Miyaura coupling of 1,4-diiodo-2,3,5,6-tetraphenylbenzene with 4-bromophenylboronic acid gives dibromo-hexaphenylbenzene, which is converted into the bis-boronic ester. Suzuki-Miyaura polymerization of the bis-boronic ester with diiodobenzene furnished polyphenylenes in a strongly sterically hindered reaction. Intramolecular Scholl reaction of the polyphenylene with FeCl₃ as oxidative reagent provides graphene nanoribbons.

Y. Fogel, L. Zhi, A. Rouhanipour, D. Andrienko, H. J. Räder, and K. Müllen, Macromolecules 2009, 42, 6878-6884 report the synthesis of a homologous series of five monodisperse ribbon-type polyphenylenes, with rigid dibenzopyrene cores in the repeat units, by microwave-assisted Diels-Alder reaction. The size of the obtained polyphenylene ribbons ranges from 132 to 372 carbon atoms in the aromatic backbone which incorporates up to six dibenzopyrene units. Because of the flexibility of the backbone and the peripheral substitution with dodecyl chains, the polyphenylene ribbons are soluble in organic solvents. In a further reaction step, ribbon-type polycyclic aromatic hydrocarbons (PAHs) are prepared by cyclodehydrogenation.

All three methods suffer from drawbacks regarding the final graphene nanoribbon.

In the first case, the resulting graphene nanoribbons are ill-defined due to the statistically arranged “kinks” in their backbone. Furthermore the molecular weight is limited due to the sensitivity of the A2B2-type polymerization approach to abberations from stochiometry. No lateral solublizing alkyl chains have been introduced into the graphene nanoribbons.

The second case suffers also from the stochiometry issue due to the underlying A2B2-stochiometry of the A2B2-type Suzuki protocol and the sterical hindrance of 1,4-diiodo-2,3,5,6-tetraphenylbenzene.

The third case makes use of a step-wise synthesis which provides very defined cut-outs from graphene nanoribbons but is impracticable for the fabrication of high-molecular weight species.

It is an object of the present invention to provide new methods for the production of graphene nanoribbons. It is a further object of the present invention to provide suitable polymeric precursors for the preparation of graphene nanoribbons, as well as methods and suitable monomeric compounds for preparing such polymeric precursors.

The problem is solved by oligophenylene monomer of general formula I

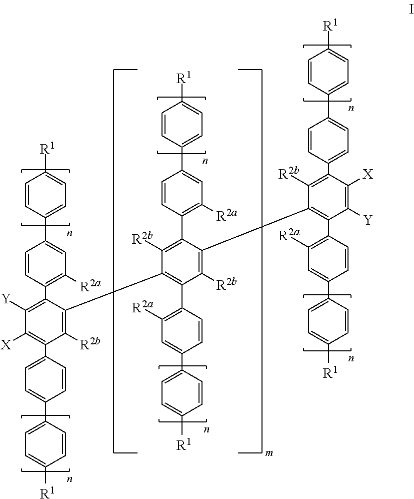

wherein

R¹ is H, halogene, —OH, —NH2, —CN, —NO₂, or a linear or branched, saturated or unsaturated C₁-C₄₀ hydrocarbon residue, which can be substituted 1- to 5-fold with halogene (F, Cl, Br, I), —OH, —NH₂, —CN and/or —NO₂, and wherein one or more CH2-groups can be replaced by —O—, —S—, —C(O)O—, —O—C(O)—, —C(O)—, —NH— or —NR³—, wherein R³ is an optionally substituted C₁-C₄₀ hydrocarbon residue, or an optionally substituted aryl, alkylaryl, alkoxyaryl, alkanoyl or aroyl residue;

R^{2a} and R^{2b} are H, or optionally one or more of the pairs of adjacent R^{2a}/R^{2b} is joined to form a single bond in a six-membered carbocycle;

m is an integer of from 0 to 3;

n is 0 or 1; and

X is halogene or trifluoromethylsulfonate, and Y is H; or X is H, and Y is halogene or trifluoromethylsulfonate.

In one embodiment of the invention, R^{2a} and R^{2b} are H. In another embodiment of the invention, each one of the pairs of adjacent R^{2a}/R^{2b} is joined to form a single bond in a six-membered carbocycle.

Preferably, m is an integer of from 0 to 2. More preferred m is 0 or 1. In one particularly preferred embodiment of the invention, m is 0. In another particularly preferred embodiment of the invention, m is 1.

Preferably, n is 0.

In one embodiment of the invention, X is halogene or trifluoromethylsulfonate, and Y is H.

A particularly preferred embodiment of the invention is the oligophenylene monomer of general formula Ia. Another particularly preferred embodiment of the invention is the oligophenylene monomer of general formula lb. Still another particularly preferred embodiment of the invention is the oligophenylene monomer of general formula Ic. Still another particularly preferred embodiment of the invention is the oligophenylene monomer of general formula Id.

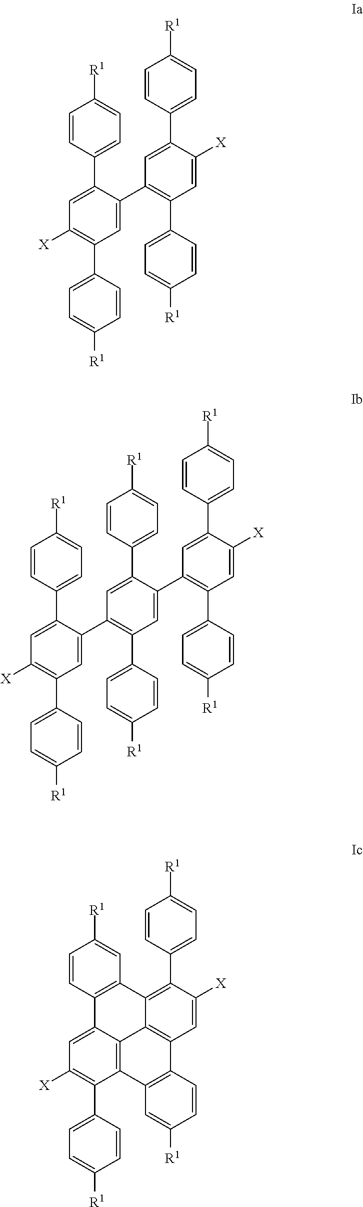

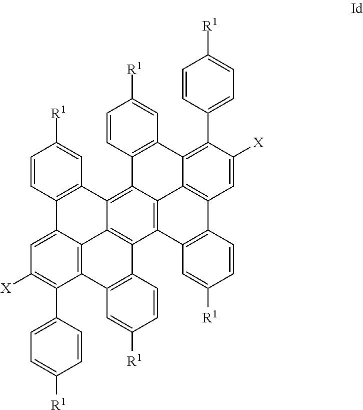

wherein

R¹ is H, halogene, —OH, —NH₂, —CN, —NO₂, or a linear or branched, saturated or unsaturated C₁-C₄₀ hydrocarbon residue, which can be substituted 1- to 5-fold with halogene (F, Cl, Br, I), —OH, —NH₂, —CN and/or —NO₂, and wherein one or more CH₂-groups can be replaced by —O—, —S—, —C(O)O—, —O—C(O)—, —C(O)—, —NH— or —NR³—, wherein R³ is an optionally substituted C₁-C₄₀ hydrocarbon residue, or an optionally substituted aryl, alkylaryl, alkoxyaryl, alkanoyl or aroyl residue; and

X is halogene or trifluoromethylsulfonate.

Preferably, X in formulae I, Ia, Ib, Ic and Id is halogene. Particularly preferred X in formulae I, Ia, Ib, Ic and Id is Cl or Br.

In another embodiment of the invention, X is H, and Y is halogene or trifluoromethylsulfonate.

A particularly preferred embodiment of the invention is the oligophenylene monomer of general formula Ie. Another particularly preferred embodiment of the invention is the oligophenylene monomer of general formula If. Still another particularly preferred embodiment of the invention is the oligophenylene monomer of general formula Ig. Still another particularly preferred embodiment of the invention is the oligophenylene monomer of general formula Ih.

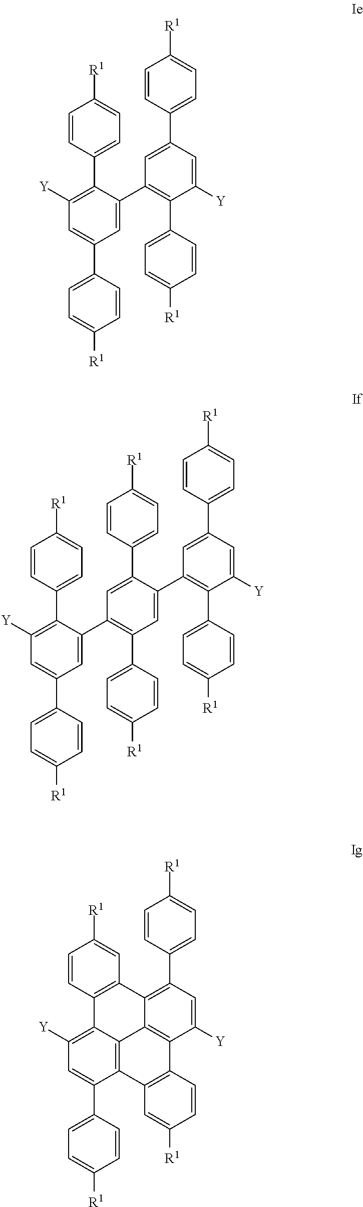

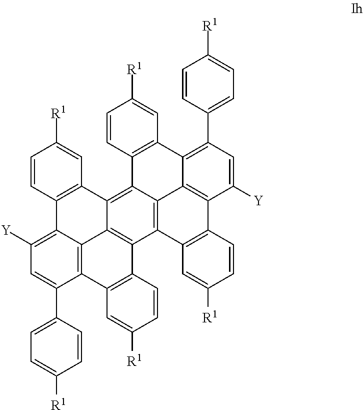

wherein

R¹ is H, halogene, —OH, —NH₂, —CN, —NO₂, or a linear or branched, saturated or unsaturated C₁-C₄₀ hydrocarbon residue, which can be substituted 1- to 5-fold with halogene (F, Cl, Br, I), —OH, —NH₂, —CN and/or —NO₂, and wherein one or more CH₂-groups can be replaced by —O—, —S—, —C(O)O—, —O —C(O)—, —C(O)—, —NH— or —NR³—, wherein R³ is an optionally substituted C₁-C₄₀ hydrocarbon residue, or an optionally substituted aryl, alkylaryl, alkoxyaryl, alkanoyl or aroyl residue; and

Y is halogene or trifluoromethylsulfonate.

Preferably, Y in formulae I, Ie, If, Ig and Ih is halogene. Particularly preferred, Y in formulae I, Ie, If, Ig and Ih is Cl or Br.

Preferably, R¹ in formulae I, Ia, Ib, Ic, Id, Ie, If, Ig and Ih is H, C₁-C₃₀ alkyl, C₁-C₃₀ alkoxy, C₁-C₃₀ alkylthio, C₂-C₃₀ alkenyl, C₂-C₃₀ alkynyl, C₁-C₃₀ haloalkyl, C₂-C₃₀ haloalkenyl or C₂-C₃₀ haloalkynyl, e.g. C₁-C₃₀ perfluoroalkyl. More preferred, R¹ in formulae I, Ia, Ib, Ic, Id, Ie, If, Ig and Ih is H, C₁-C₃₀ alkyl or C₁-C₃₀ alkoxy. Most preferred, R¹ in formulae I, Ia, Ib, Ic, Id, Ie, If, Ig and Ih is H or C₁-C₃₀ alkyl.

C₁-C₃₀ alkyl can be linear or branched, where possible. Examples are methyl, ethyl, n-propyl, isopropyl, n-butyl, sec.-butyl, isobutyl, tert.-butyl, n-pentyl, 2-pentyl, 3-pentyl, 2,2-dimethylpropyl, 1,1,3,3-tetramethylpentyl, n-hexyl, 1-methylhexyl, 1,1,3,3,5,5-hexamethylhexyl, n-heptyl, isoheptyl, 1,1,3,3-tetramethylbutyl, 1-methylheptyl, 3-methylheptyl, n-octyl, 1,1,3,3-tetramethylbutyl and 2-ethylhexyl, n-nonyl, decyl, undecyl, dodecyl, tridecyl, tetradecyl, pentadecyl, hexadecyl, heptadecyl, octadecyl, eicosyl, heneicosyl, docosyl, tetracosyl or pentacosyl.

C₁-C₃₀ alkoxy groups are straight-chain or branched alkoxy groups, e.g. methoxy, ethoxy, n-propoxy, isopropoxy, n-butoxy, sec-butoxy, tert-butoxy, amyloxy, isoamyloxy, tert-amyloxy, heptyloxy, octyloxy, isooctyloxy, nonyloxy, decyloxy, undecyloxy, dodecyloxy, tetradecyloxy, pentadecyloxy, hexadecyloxy, heptadecyloxy or octadecyloxy.

The term “alkylthio group” means the same groups as the alkoxy groups, except that the oxygen atom of the ether linkage is replaced by a sulfur atom.

C₂-C₃₀ alkenyl groups are straight-chain or branched alkenyl groups, such as e.g. vinyl, allyl, methallyl, isopropenyl, 2-butenyl, 3-butenyl, isobutenyl, n-penta-2,4-dienyl, 3-methyl-but-2-enyl, n-oct-2-enyl, n-dodec-2-enyl, isododecenyl, n-dodec-2-enyl or n-octadec-4-enyl.

C₂-C₃₀ alkynyl is straight-chain or branched and may be unsubstituted or substituted, such as, for example, ethynyl, 1-propyn-3-yl, 1-butyn-4-yl, 1-pentyn-5-yl, 2-methyl-3-butyn-2-yl, 1,4-pentadiyn-3-yl, 1,3-pentadiyn-5-yl, 1-hexyn-6-yl, cis-3-methyl-2-penten-4-yn-1-yl, trans-3-methyl-2-penten-4-yn-1-yl, 1,3-hexadiyn-5-yl, 1-octyn-8-yl, 1-nonyn-9-yl, 1-decyn-10-yl, or 1-tetracosyn-24-yl.

C₁-C₃₀-perfluoroalkyl is a branched or unbranched radical such as for example —CF₃, —CF₂CF₃, —CF₂CF₂CF₃, —CF(CF₃)₂, —(CF₂)₃CF₃ or —C(CF₃)₃.

The terms “haloalkyl, haloalkenyl and haloalkynyl” mean groups given by partially or wholly substituting the abovementioned alkyl group, alkenyl group and alkynyl group with halogen.

C₂-C₃₀ acyl is straight-chain or branched and may be saturated or unsaturated, such as, for example, ethanoyl, propanoyl, isobutanoyl, n-butanoyl, pentanoyl, hexanoyl, heptanoyl, octanoyl, nonanoyl, decanoyl or dodecanoyl.

Aryl is usually C₆-C₃₀ aryl, which optionally can be substituted, such as, for example, phenyl, 4-methylphenyl, 4-methoxyphenyl, naphthyl, biphenylyl, terphenylyl, pyrenyl, fluorenyl, phenanthryl, anthryl, tetracyl, pentacyl or hexacyl.

The problem is further solved by polymeric precursors for the preparation of graphene nanoribbons obtainable from the oligophenylene monomers of general formula I as defined above.

The polymeric precursors for the preparation of graphene nanoribbons have thereby repeating units of general formulae II or II′,

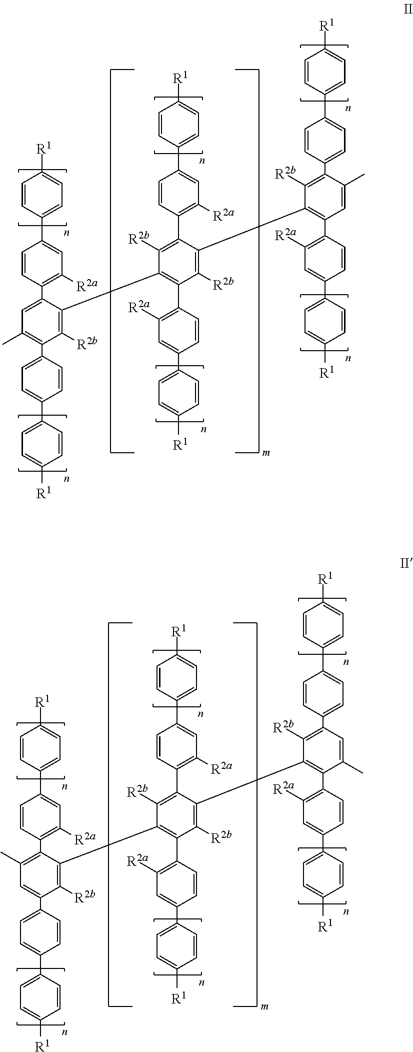

wherein R¹, R₂ₐ, R_{2b}, m and n are as defined above.

Preferable polymeric precursors for the preparation of graphene nanoribbons are obtained by polymerization of oligophenylene monomers of general formulae Ia, Ib, Ic, Id, Ie, If, Ig or Ih,

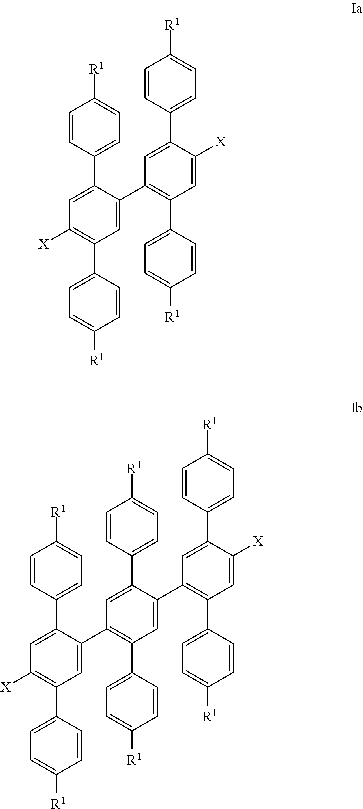

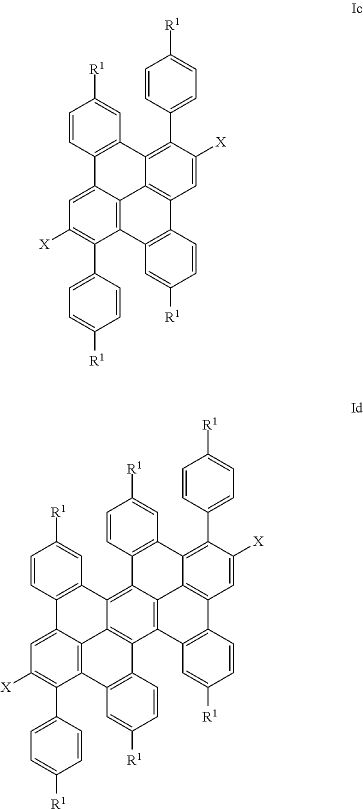

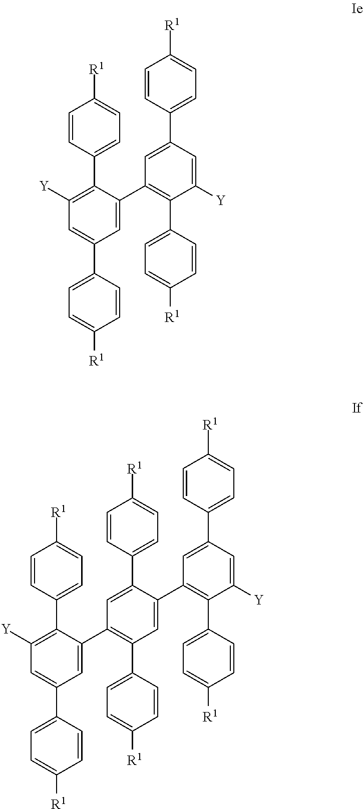

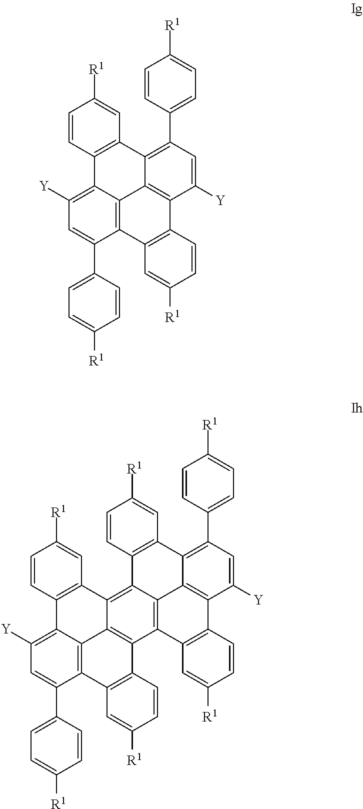

wherein R¹, X and Y are as defined above.

In one embodiment of the invention, the polymeric precursors for the preparation of graphene nanoribbons are obtained by polymerization of oligophenylene monomers of general formulae Ia, Ib, Ic or Id. In a preferred embodiment of the invention, the polymeric precursors are obtained by polymerization of oligophenylene monomers of general formulae Ia or Ib. In a particularly preferred embodiment of the invention, the polymeric precursors are obtained by polymerization of oligophenylene monomers of general formula Ia. In a particularly preferred embodiment of the invention, the polymeric precursors are obtained by polymerization of oligophenylene monomers of general formula Ib.

In another embodiment of the invention, the polymeric precursors for the preparation of graphene nanoribbons are obtained by polymerization of oligophenylene monomers of general formulae Ie, If, Ig or Ih. In a preferred embodiment of the invention, the polymeric precursors are obtained by polymerization of oligophenylene monomers of general formulae Ie or If. In a particularly preferred embodiment of the invention, the polymeric precursors are obtained by polymerization of oligophenylene monomers of general formula Ie. In a particularly preferred embodiment of the invention, the polymeric precursors are obtained by polymerization of oligophenylene monomers of general formula If. In a preferred embodiment of the invention, the polymeric precursors are obtained by polymerization of oligophenylene monomers of general formulae Ig or Ih. In a particularly preferred embodiment of the invention, the polymeric precursors are obtained by polymerization of oligophenylene monomers of general formula Ig. In a particularly preferred embodiment of the invention, the polymeric precursors are obtained by polymerization of oligophenylene monomers of general formula Ih.

The preferable polymeric precursors for the preparation of graphene nanoribbons have repeating units of general formulae IIa, IIb, IIc, IId, IIe, IIf, IIg or IIh,

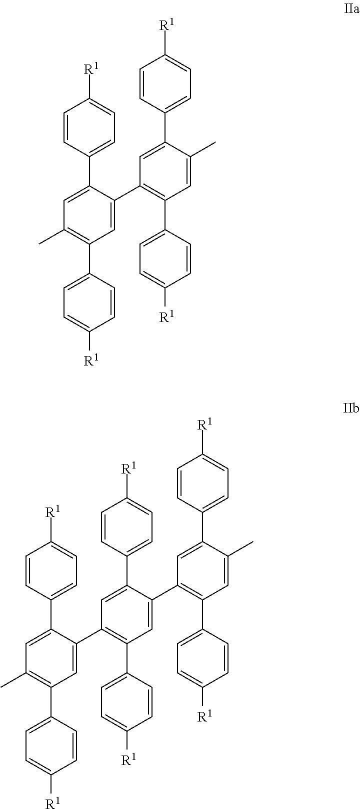

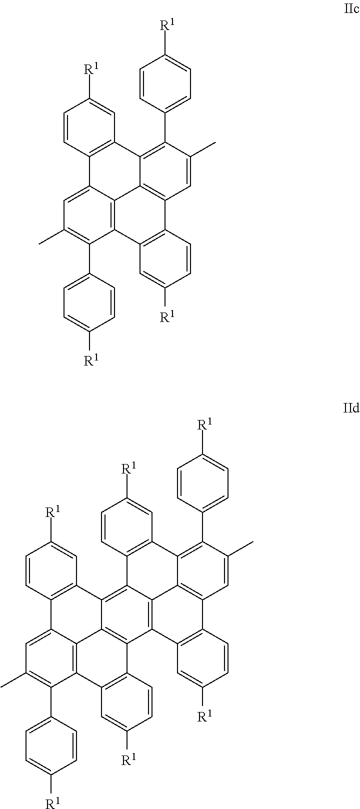

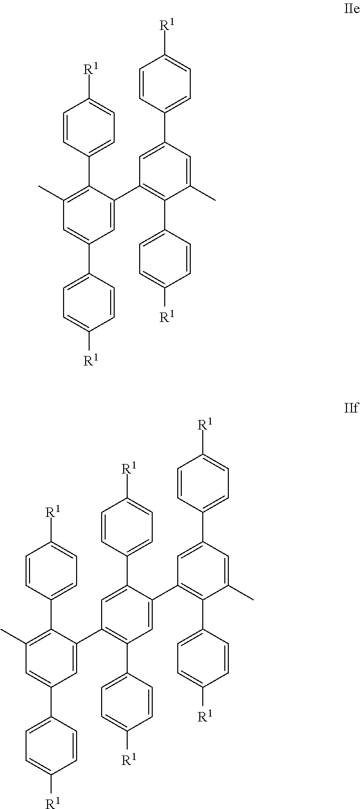

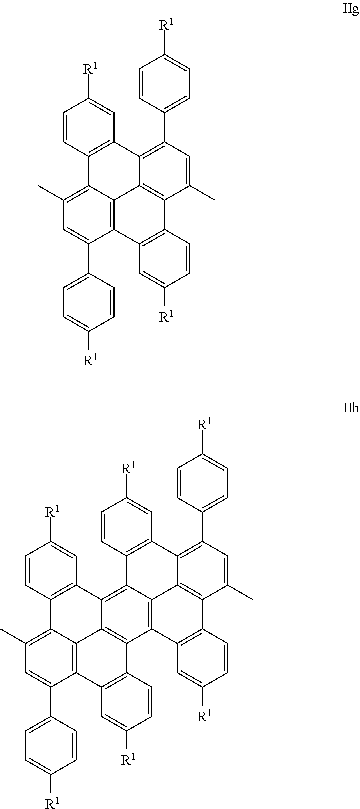

wherein R¹ is as defined above.

In one embodiment of the invention, the polymeric precursors have repeating units of general formulae IIa, IIb, IIc or IId, IIe, IIf, IIg or IIh. In a preferred embodiment of the invention, the polymeric precursors have repeating units of general formulae IIa or IIb. In a particularly preferred embodiment of the invention, the polymeric precursors have repeating units of general formula IIa. In a particularly preferred embodiment of the invention, the polymeric precursors have repeating units of general formula IIb.

In another embodiment of the invention, the polymeric precursors have repeating units of general formulae IIe, IIf, IIg or IIh. In a preferred embodiment of the invention, the polymeric precursors have repeating units of general formulae IIe or IIf. In a particularly preferred embodiment of the invention, the polymeric precursors have repeating units of general formula IIe. In a particularly preferred embodiment of the invention, the polymeric precursors have repeating units of general formula IIf. In a preferred embodiment of the invention, the polymeric precursors have repeating units of general formulae IIg or IIh. In a particularly preferred embodiment of the invention, the polymeric precursors have repeating units of general formula IIg. In a particularly preferred embodiment of the invention, the polymeric precursors have repeating units of general formula IIh.

In one further aspect of the present invention, polymeric precursors having repeating units of general formulae II or II′ are prepared from oligophenylene monomers of general formula I by Yamamoto-polycondensation reaction according to Scheme 1. The reaction can be carried out e.g. in dimethylformamide (DMF) or in a mixture of toluene and DMF. The catalyst can be prepared from a stoichiometric mixture of bis(cyclooctadiene)nickel(0), 1,5-cyclooctadiene and 2,2′-bipyridine e.g. in a mixture of toluene and DMF. Preferably, the reaction is carried out using the dichloro (X or Y in formula I is Cl) or the dibromo-compound (X or Y in formula I is Br). Depending on the substituents X or Y, the polycondensation reaction is carried out at temperatures of from 50 to 110° C. Preferably, the polycondensation reaction is carried out at temperatures of from 70 to 90° C.

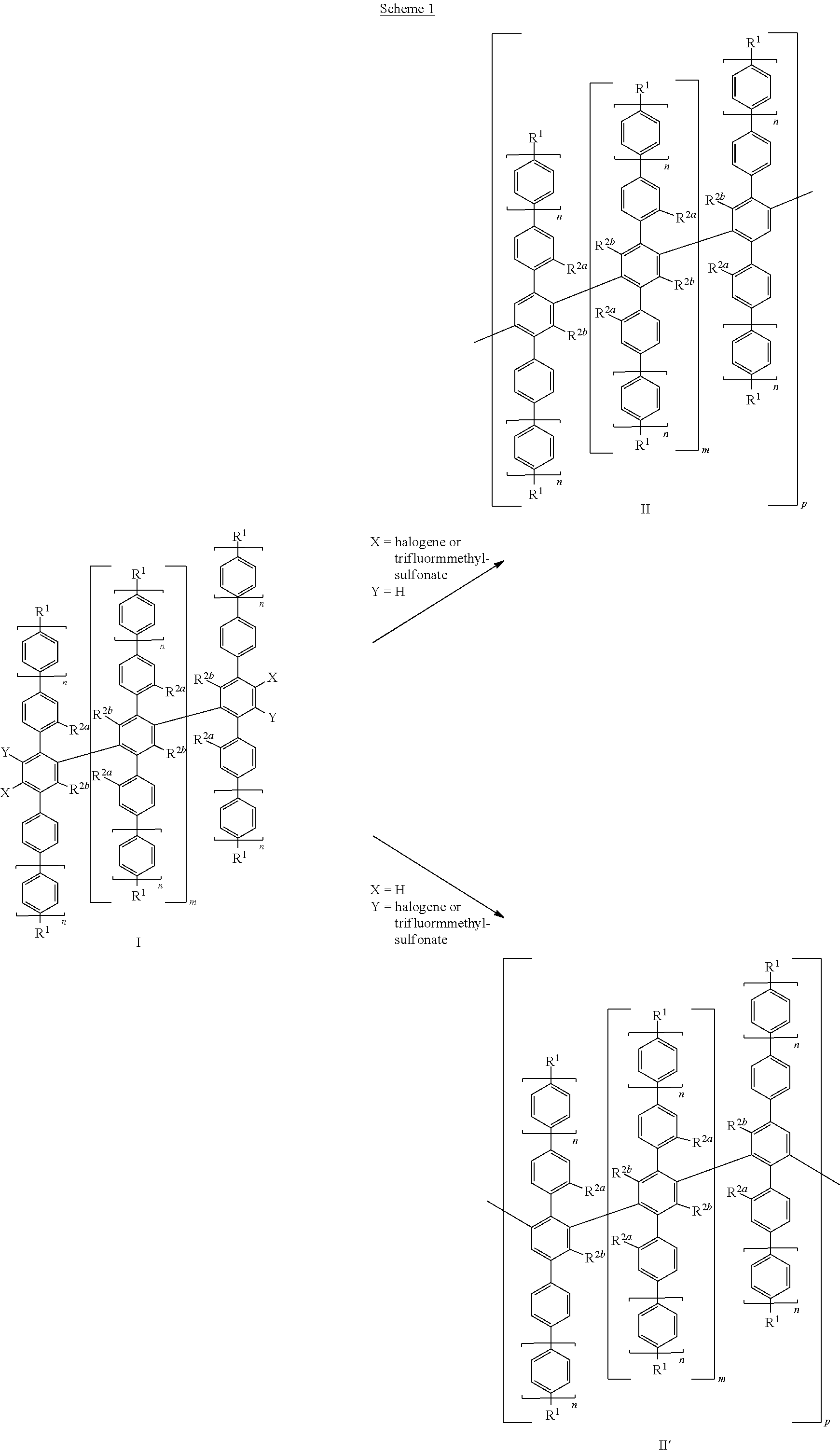

wherein R¹, R^{2a}, R^{2b}, m and n are as defined above.

The quenching of the reaction and the decomposition of nickel residues can be achieved by carefully dropping the reaction mixture into dilute methanolic hydrochloric acid. A white precipitate instantly formed which can be collected by filtration. The material can be re-dissolved in DCM, filtered and re-precipitated. The number of repeating units p varies in general from 2 to 1000.

The preferable polymeric precursors having repeating units of general formulae IIa, IIb, IIc, IId, IIe, IIf, IIg or IIh are prepared from oligophenylene monomers of general formulae Ia, Ib, Ic, Id, Ie, If, Ig or Ih using the same methodology.

In a particular aspect of the present invention, graphene nanoribbons are obtained by cyclodehydrogenation of polymeric precursors of general formulae II or II′. Preferably, the graphene nanoribbons are obtained by cyclodehydrogenation of polymeric precursors of general formulae IIa, IIb, IIc, IId, IIe, IIf, IIg or IIh.

In one embodiment of the invention, the graphene nanoribbons are obtained by cyclodehydrogenation of polymeric precursors of general formulae IIa, IIb, IIc, IId, IIe, IIf, IIg or IIh. In a preferred embodiment of the invention, the graphene nanoribbons are obtained by cyclodehydrogenation of polymeric precursors of general formulae IIa or IIb. In a particularly preferred embodiment of the invention, the graphene nanoribbons are obtained by cyclodehydrogenation of polymeric precursors of general formula IIa. In a particularly preferred embodiment of the invention, the graphene nanoribbons are obtained by cyclodehydrogenation of polymeric precursors of general formula IIb.

In another embodiment of the invention, the graphene nanoribbons are obtained by cyclodehydrogenation of polymeric precursors of general formulae IIe, IIf, IIg or IIh. In a preferred embodiment of the invention, the graphene nanoribbons are obtained by cyclodehydrogenation of polymeric precursors of general formulae IIe or IIf. In a particularly preferred embodiment of the invention, the graphene nanoribbons are obtained by cyclodehydrogenation of polymeric precursors of general formula IIe. In a particularly preferred embodiment of the invention, the graphene nanoribbons are obtained by cyclodehydrogenation of polymeric precursors of general formula IIf. In a preferred embodiment of the invention, the graphene nanoribbons are obtained by cyclodehydrogenation of polymeric precursors of general formulae IIg or IIh. In a particularly preferred embodiment of the invention, the graphene nanoribbons are obtained by cyclodehydrogenation of polymeric precursors of general formula IIg. In a particularly preferred embodiment of the invention, the polymeric precursors have repeating units of general formula IIh.

The graphene nanoribbons are preferably prepared in a solution process. The preparation of graphene nanoribbons from the high-molecular weight polymeric precursors can be performed using ferric chloride as oxidant in a mixture of dichloromethane (DCM) and nitromethane.

Alternatively, the preparation of graphene nanoribbons can be carried out using phenyliodine(III) bis(trifluoroacetate) (PIFA) and BF₃ etherate in anhydrous DCM. It is known that PIFA when activated by a Lewis acid readily reacts with a broad range of substrates to give biaryl products in excellent yields (Takada, T.; Arisawa, M.; Gyoten, M.; Hamada, R.; Tohma, H.; Kita, Y. J. Org. Chem. 1998, 63, 7698-7706). Furthermore, it was recently applied to the synthesis of triphenylenes (King, B. T.; Kroulik, J.; Robertson, C. R.; Rempala, P.; Hilton, C. L.; Korinek, J. D.; Gortari, L. M. J. Org. Chem. 2007, 72, 2279-2288.) and hexa-peri-hexabenzocoronene (HBC) derivatives (Rempala, P.; Kroulik, J.; King, B. T. J. Org. Chem. 2006, 71, 5067-5081.). Importantly, undesired chlorination which is frequently observed when applying ferric chloride can be ruled out by this procedure. The reactions with PIFA/BF₃.Et₂O (2.50 bond equivalents) were started at −60° C. and the thermal level was slowly raised to room temperature after 2 h. The reaction was allowed to proceed for additional 24 h after what it was stopped by addition of methanol. Graphitic insoluble graphene nanoribbons are obtained in quantitative yield.

In general, the molecular weight of the graphene nanoribbons obtained by cyclodehydrogenation in the solution process varies from 10 000 to 1 000 000 g/mol, preferably from 20 000 to 200 000 g/mol.

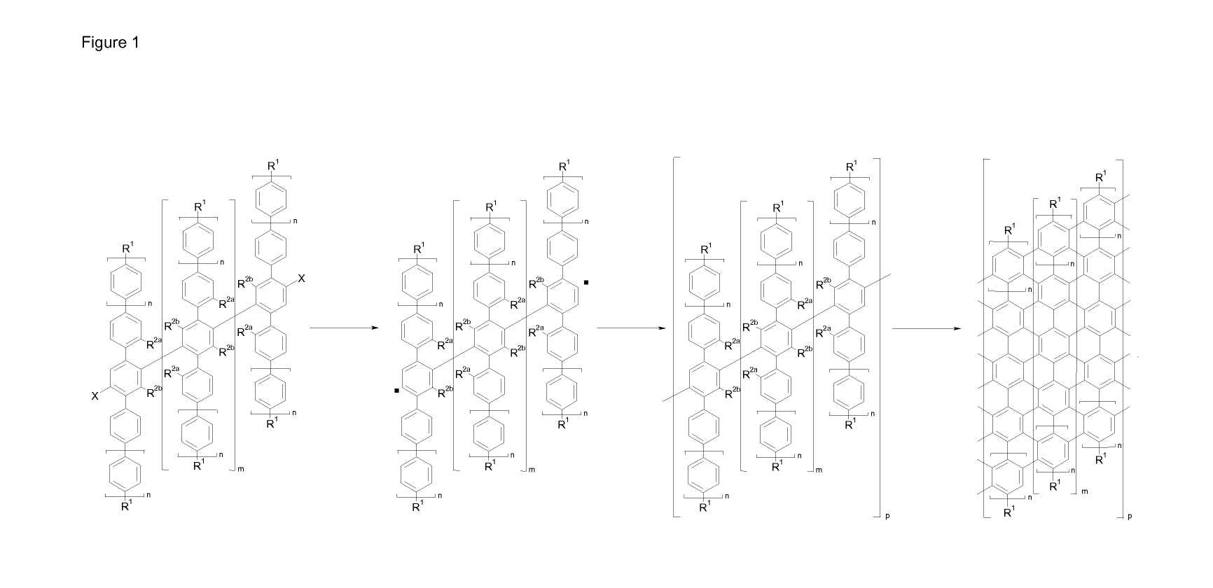

Covalently bonded two-dimensional molecular arrays can be efficiently studied by scanning tunneling microscope (STM) techniques. Examples of surface-confined covalent bond formation involve Ullmann coupling, imidization, crosslinking of porphyrins and oligomerization of heterocyclic carbenes and polyamines. A chemistry-driven protocol for the direct growth of grapene nanoribbons and graphene networks on surfaces has been very recently established by the groups of Müllen (MPI-P Mainz, Germany) and Fasel (EMPA Dübendorf, Switzerland) (Bieri, M.; Treier, M.; Cai, J.; Aït-Mansour, K.; Ruffieux, P.; Gröning, O., Gröning, P.; Kastler, M.; Rieger, R.; Feng, X.; Müllen, K.; Fasel, R.; Chem. Commun. 2009, 45, 6919; Bieri, M.; Nguyen, M. T.; Gröning, 0.; Cai, J.; Treier, M.; Aït-Mansour, K.; Ruffieux, P.; Pignedoli, C. A.; Passerone, D.; Kastler, M.; Müllen, K.; Fasel, R.; J. Am. Chem. Soc. 2010, 132, 16669; Treier, M.; Pignedoli, C. A.; Laino, T.; Rieger, R.; Müllen, K.; Passerone, D.; Fasel, R. Nature Chemistry 2011, 3, 61; Cai, J.; Ruffieux, P.; Jaafar, R.; Bieri, M.; Braun, T.; Blankenburg, S.; Muoth, M.; Seitsonen, A. P.; Saleh, M.; Feng, X.; Müllen, K.; Fasel, R. Nature 2010, 466, 470-473.). Without being bound by theory it can be concluded from these studies that the nanoribbon formation on the metal surface proceeds via a radical pathway (FIG. 1). After deposition of the functionalized monomer on the surface via ultra high vacuum (UHV) sublimation (10⁻¹¹ to 10⁻⁵ mbar, preferably 10⁻¹⁰ to 10⁻⁷ mbar) dehalogenation is believed to occur upon thermal activation by annealing to 100-200° C. This generates biradical species that diffuse on the surface and couple to each other resulting in the formation of carbon-carbon bonds. These radical addition reactions proceed at intermediate thermal levels (100 to 250° C., preferably 150 to 220° C.) and are the prerequisite for the subsequent cyclodehydrogenation at higher temperatures (350 to 450° C., preferably 380 to 420° C.). Only if polymeric species of sufficient molecular weight are formed during the first stage, the full graphitization of the molecules will proceed subsequently with the thermal desorption of the material from the surface being avoided.

For UHV surface-assisted polymerization and cyclodehydrogenation, functional monomers of sufficiently high rigidity and planarity are needed which assist in the flat orientation on the metal substrate. Also, the method allows for the topological tailoring of the graphene nanoribbons as their shape is determined by the functionality pattern and geometry of the precursor monomers. Solubilizing alkyl chains are not needed in the monomer design as no solvent-based process is involved in this surface-bound protocol.

In another particular aspect of the present invention, graphene nanoribbons are prepared by direct growth of the graphene nanoribbons on surfaces by polymerization of oligophenylene monomers of general formula I as defined above and cyclodehydrogenation. Preferably, the graphene nanoribbons are prepared by polymerization of oligophenylene monomers of general formulae Ia, Ib, Ic, Id, Ie, If, Ig or Ih as defined above, and cyclodehydrogenation.

In one embodiment of the invention, the graphene nanoribbons are prepared by polymerization of oligophenylene monomers of general formulae Ia, Ib, Ic or Id. In a preferred embodiment of the invention, the graphene nanoribbons are prepared by polymerization of oligophenylene monomers of general formulae Ia or Ib. In a particularly preferred embodiment of the invention, the graphene nanoribbons are prepared by polymerization of oligophenylene monomers of general formula Ia. In a particularly preferred embodiment of the invention, the graphene nanoribbons are prepared by polymerization of oligophenylene monomers of general formula Ib.

In another embodiment of the invention, the graphene nanoribbons are prepared by polymerization of oligophenylene monomers of general formulae Ie, If, Ig or Ih. In a preferred embodiment of the invention, the graphene nanoribbons are prepared by polymerization of oligophenylene monomers of general formulae Ie or If. In a particularly preferred embodiment of the invention, the graphene nanoribbons are prepared by polymerization of oligophenylene monomers of general formula Ie. In a particularly preferred embodiment of the invention, the graphene nanoribbons are prepared by polymerization of oligophenylene monomers of general formula If. In a preferred embodiment of the invention, the graphene nanoribbons are prepared by polymerization of oligophenylene monomers of general formulae Ig or Ih. In a particularly preferred embodiment of the invention, the graphene nanoribbons are prepared by polymerization of oligophenylene monomers of general formula Ig. In a particularly preferred embodiment of the invention, the graphene nanoribbons are prepared by polymerization of oligophenylene monomers of general formula Ih.

In general, the molecular weight of the graphene nanoribbons obtained by direct growth of the graphene nanoribbons on surfaces by polymerization of oligophenylene monomers and subsequent cyclodehydrogenation varies from 2 000 to 1 000 000 g/mol, preferably from 4 000 to 100 000 g/mol.

In the following, several synthetic routes to oligophenylene monomers that can be subsumed under general formula I are presented. If not stated otherwise, R¹, R^{2a}, R^{2b}, m, n, X and Y have the meaning as defined above.

The oligophenylene monomer of formula I, wherein R^{2a} and R^{2b} are H, and Y is H, is hereinafter referred to as I-1. The oligophenylene monomer I-1 can be synthesized according to Schemes 2 to 3 below. The reaction conditions and solvents used are purely illustrative, of course other conditions and solvents can also be used and will be determined by the person skilled in the art. The synthesis of the oligophenylene monomer I-1 starts from commercially available 1,4-diiodobenzene 1 (Scheme 2). In the first step of the reaction sequence 1-4-diiodobenzene 1 is dissolved in tetrahydrofurane (THF). After the addition of 2.2 equivalents of trimethylsilylchloride (trimethylsilyl=TMS), the reaction mixture is cooled to −78° C. and treated with 2.2 equivalents of diisopropylamide to yield 1,4-diiodo-2,5-bis(trimethylsilyl)benzene. Depending on the desired width of the graphene nanoribbons, 1,4-diiodo-2,5-bis(trimethylsilyl)benzene is either reacted with commercially available 4-substituted phenylboronic acid or commercially available 4′-substituted biphenylboronic acid in a Suzuki reaction. Both the 4-substituted phenylboronic acid and the 4′-substituted biphenylboronic acid are hereinafter referred to as boronic acid 2.

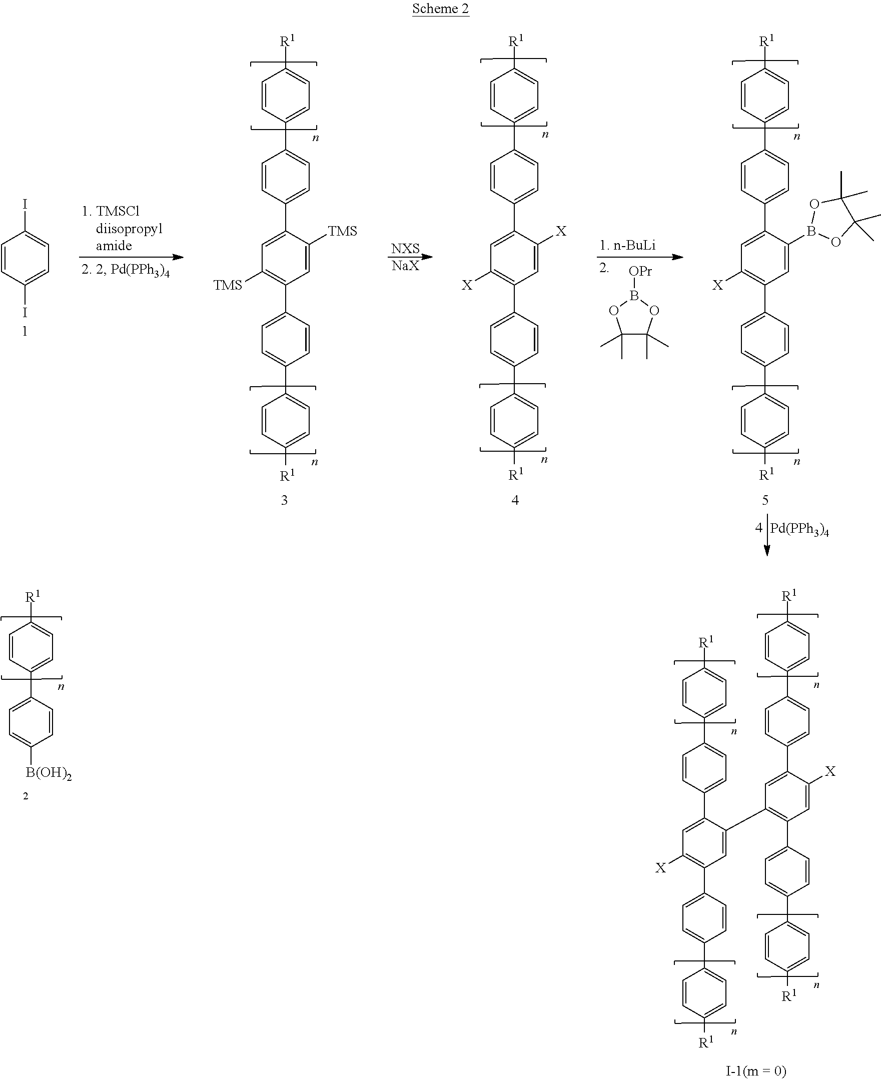

The reaction of 1,4-diiodo-2,5-bis(trimethylsilyl)-benzene with boronic acid 2 can e.g. be performed at an elevated temperature in a reaction mixture of tetrahydrofurane (THF), ethanol and water in the presence of potassium carbonate and catalytic amounts of tetrakis(triphenylphosphine)palladium(0) (Pd(PPh₃)₄). Preferably, 2 to 5 equivalents of the boronic acid 2 are used. The so obtained substituted 1,4-bis(oligophenylenyl)-2,5-bis(trimethylsilyl)benzene 3 is further reacted with N-halosuccinimide (NXS) and sodium halogenide (NaX) under reflux in a mixture of THF and methanol to yield the substituted 1,4-bis(oligophenylenyI)-2,5-dihalo-benzene 4. The arylboronic acid pinacol ester 5 can be prepared from the substituted 1,4-bis(oligophenylenyl)-2,5-dihalo-benzene 4 using n-butyl lithium (n-BuLi) and (prop-2-yloxy)boronic acid pinacol ester in THF. Subsequently, Suzuki cross coupling of arylboronic acid pinacol ester 5 with substituted 1,4-bis(oligophenylenyI)-2,5-dihalo-benzene 4 in the presence of potassium carbonate and catalytic amounts of tetrakis(triphenylphosphine)palladium(0) (Pd(PPh₃)₄) in a mixture of toluene and THF yields the oligophenylene monomer I-1(m=0).

If desired, the oligophenylene monomer I-1(m=0) can be further reacted to oligophenylene monomer I-1 by repeating the last two synthesis steps in times (Scheme 3). For example, the preferred oligophenylene monomers Ia and Ib can be synthesized in the described manner.

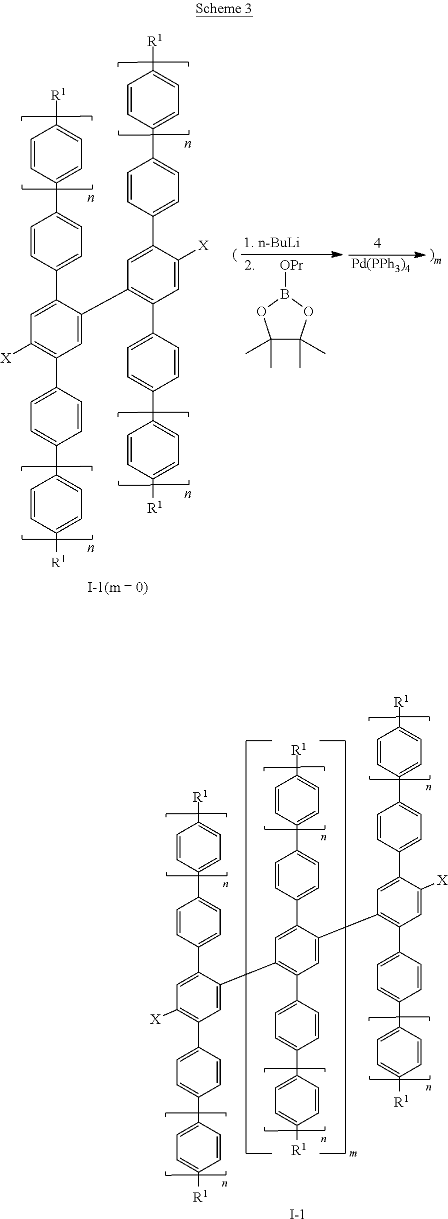

The oligophenylene monomer of formula I, wherein each one of the pairs of adjacent R^{2a}/R^{2b} is joined to form a single bond in a six-membered carbocycle, Y is H, and m is 0, is hereinafter referred to as I-2. The oligophenylene monomer I-2 can be prepared as shown in Schemes 4 to 8 below. Again, the reaction conditions and solvents are purely illustrative. A person skilled in the art can determine other conditions and solvents that are equally suitable as the ones disclosed hereinafter.

The synthesis of the oligophenylene monomer I-2 starts from commercially available 5-bromo-2-chlorophenyl methyl ether 6 (Scheme 4). In the first step of the reaction sequence, the 5-bromo-2-chlorophenyl methyl ether 6 is subjected to iodination using iodine and AgNO₃ in methanol at 50° C. to yield 5-bromo-2-chloro-4-iodophenyl methyl ether 7. Negishi cross coupling can be used for the build-up of the substituted biphenyl 9. At first, Zinc-Copper couple (commercially available under this name, for example from Sigma Aldrich) and 1,2-dibromoethane are heated under reflux in tetrahydrofurane for 30 min. After cooling to room temperature, trimethylsilyl chloride is added and stirred at room temperature for 30 min. Next, a solution of 5-bromo-2-chloro-4-iodophenyl methyl ether 7 in dimethylformamide (DMF) is added to the tetrahydrofurane solution of Zinc-Copper couple and heated under reflux. The zinc organyl 8 obtained as an intermediate is then transferred directly into the solution of 5-bromo-2-chloro-4-iodophenyl methyl ether 7 and catalytic amounts of Pd(PPh₃)₄ in tetrahydrofurane and heated under reflux to yield substituted biphenyl 9.

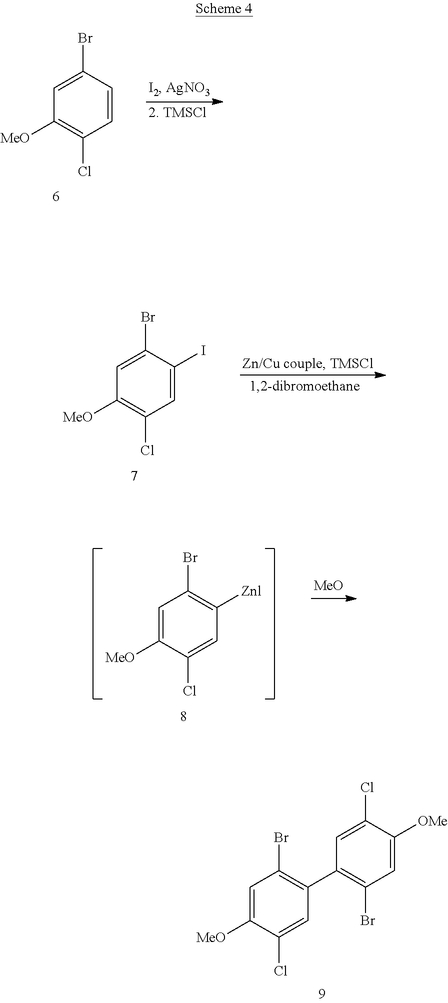

Substituted biphenyl 9 is lithiated by slowly adding exactly 2.0 equivalents of n-butyl lithium in hexane into a solution of substituted biphenyl 9 in diethyl ether at −78° C., and then treated with trimethylsilyl chloride to yield substituted biphenyl 10 (Scheme 5). Substituted oligophenyl 11 is synthesized by heating a toluene solution of substituted biphenyl 10 and boronic acid 2 at 100° C. in the presence of K₃PO₄ and catalytic amounts of a 1:2.5 ratio of Pd(OAc)₂ and 2-dicyclohexylphosphino-2′,6′-dimethoxybiphenyl (SPhos).

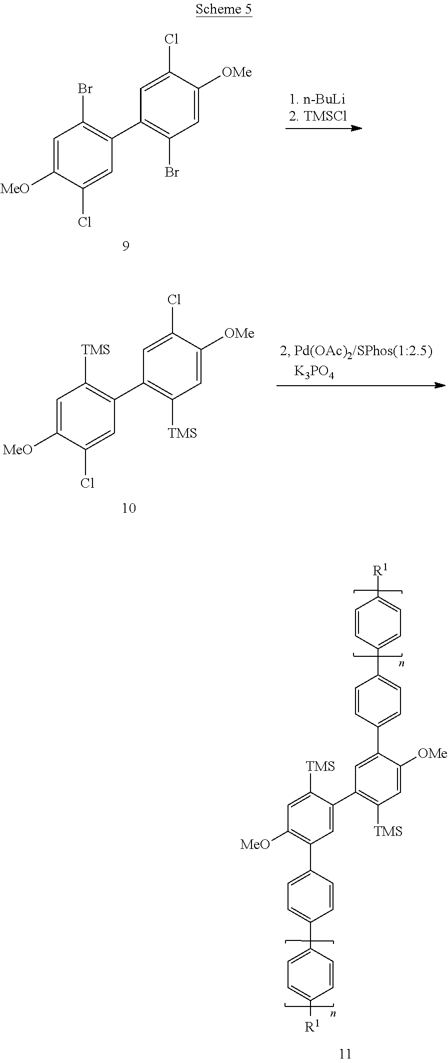

Substituted oligophenyl 11 is converted to substituted oligophenyl 12 by adding drop-wise a solution of 4.0 equivalents of iodine monochloride (ICl) in CH₂Cl₂ to a solution of substituted oligophenyl 11 in CHCl₃ and stirring at room temperature (Scheme 6). Next, substituted oligophenyl 12 is subjected to Suzuki coupling by heating to reflux a solution of substituted oligophenyl 12 and boronic acid 13 in a 4:1:1 mixture of toluene, ethanol, and water under in the presence of K₂CO₃ and a catalytic amounts of Pd(PPh₃)₄ to afford substituted oligophenyl 14.

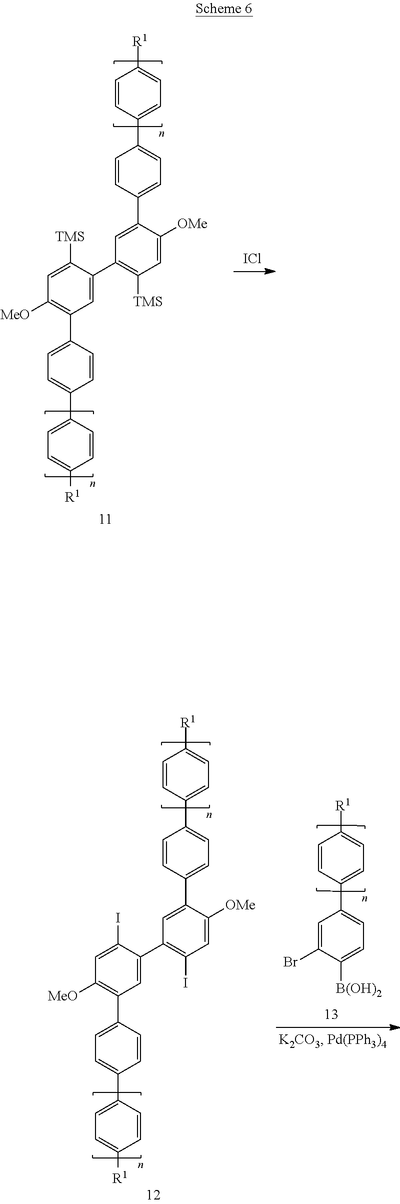

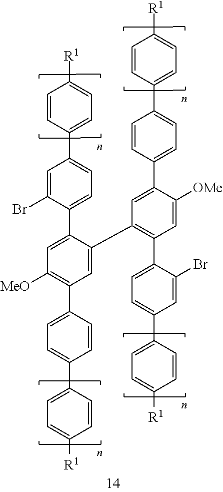

Substituted oligophenyl 14 is subjected to Pd-catalyzed intramolecular arylation by heating at 160° C. in N,N-dimethylacetamide (DMA) under the existence of 1,8-diazabicyclo[5.4.0]undec-7-ene (DBU) and catalytic amounts of Pd(OAc)₂ and Buchwald ligand such as 2-dicyclohexylphosphino-2′,6′-dimethoxybiphenyl to yield substituted dibenzonaphthacene 14 (Scheme 7). For the demethylation of substituted dibenzonaphthacene 15 BBr₃ is added to a solution of 15 in CH₂Cl₂at 0° C. and then the reaction mixture is slowly warmed to room temperature to obtain substituted dibenzonaphthacene 16.

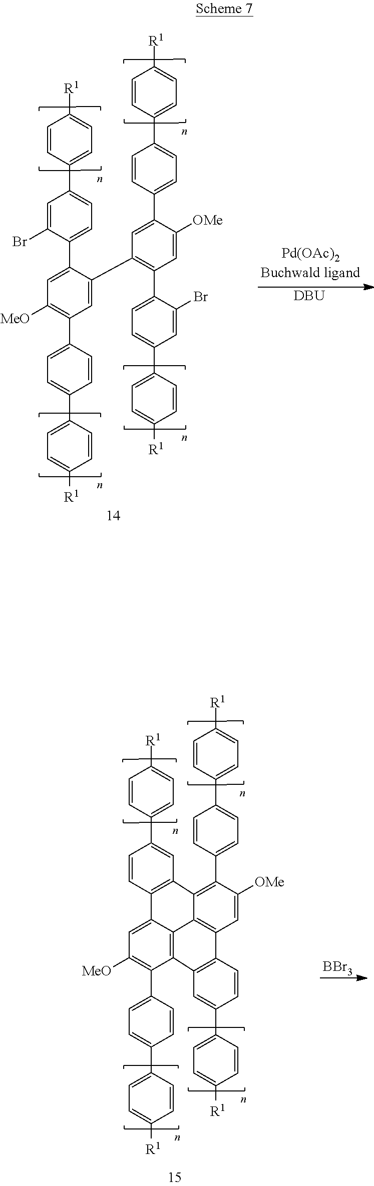

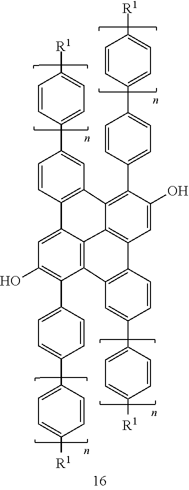

Substituted dibenzonaphthacene 16 is converted to substituted dibenzonaphthacene 17 by adding trifluoromethanesulfonic anhydride (Tf₂O) dropwise to a solution of dibenzonaphthacene 16 and Et₃N in CH₂Cl₂ at 0° C. and then stirring at room temperature (Scheme 8). Substituted dibenzonaphthacene 17 is next heated to reflux with Na in EtOAc to afford substituted dibenzonaphthacene I-2.

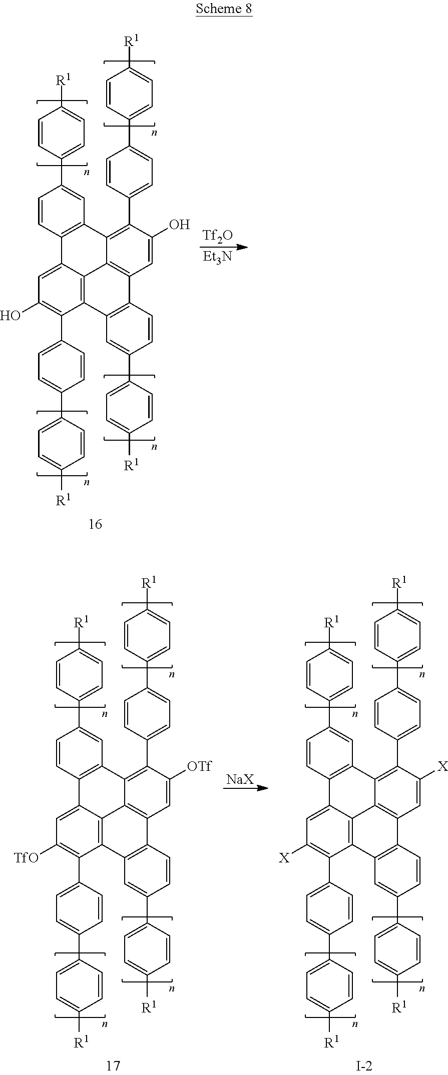

For example, the preferred oligophenylene monomer Ic can be synthesized as described in Schemes 4 to 8.

Various articles of manufacture including electronic devices, optical devices, and optoelectronic devices, such as field effect transistors (e.g., thin film transistors), photovoltaics, organic light emitting diodes (OLEDs), complementary metal oxide semiconductors (CMOSs), complementary inverters, D flip-flops, rectifiers, and ring oscillators, that make use of the graphene nanoribbons disclosed herein also are within the scope of the present invention as are methods of making the same.

The present invention, therefore, further provides methods of preparing a semiconductor material exhibiting a well-defined electronic band gap that can be tailored to specific applications by the choice of molecular precursor. The methods can include preparing a composition that includes one or more of the compounds of the invention disclosed herein dissolved or dispersed in a liquid medium such as a solvent or a mixture of solvents, depositing the composition on a substrate to provide a semiconductor material precursor, and processing (e.g., heating) the semiconductor precursor to provide a semiconductor material (e.g., a thin film semiconductor) that includes one or more of the compounds disclosed herein. In various embodiments, the liquid medium can be an organic solvent, an inorganic solvent such as water, or combinations thereof. In some embodiments, the composition can further include one or more additives independently selected from detergents, dispersants, binding agents, compatibilizing agents, curing agents, initiators, humectants, antifoaming agents, wetting agents, pH modifiers, biocides, and bacteriostats. For example, surfactants and/or polymers (e.g., polystyrene, polyethylene, poly-alpha-methylstyrene, polyisobutene, polypropylene, polymethyl-methacrylate, and the like) can be included as a dispersant, a binding agent, a compatibilizing agent, and/or an antifoaming agent. In some embodiments, the depositing step can be carried out by printing, including inkjet printing and various contact printing techniques (e.g., screen-printing, gravure printing, offset printing, pad printing, lithographic printing, flexographic printing, and microcontact printing). In other embodiments, the depositing step can be carried out by spin coating, drop-casting, zone casting, dip coating, blade coating, spraying or vacuum filtration.

The present invention further provides articles of manufacture such as the various devices described herein that include a composite having a semiconductor material of the present invention and a substrate component and/or a dielectric component. The substrate component can be selected from doped silicon, an indium tin oxide (ITO), ITO-coated glass, ITO-coated polyimide or other plastics, aluminum or other metals alone or coated on a polymer or other substrate, a doped polythiophene, and the like. The dielectric component can be prepared from inorganic dielectric materials such as various oxides (e.g., SiO₂, Al₂O₃, HfO₂), organic dielectric materials such as various polymeric materials (e.g., polycarbonate, polyester, polystyrene, polyhaloethylene, polyacrylate), and self-assembled superlattice/self-assembled nanodielectric (SAS/SAND) materials (e.g., described in Yoon, M-H. et al., PNAS, 102 (13): 4678-4682 (2005)), as well as hybrid organic/inorganic dielectric materials (e.g., described in US 2007/0181961A1). The composite also can include one or more electrical contacts. Suitable materials for the source, drain, and gate electrodes include metals (e.g., Au, Al, Ni, Cu), transparent conducting oxides (e.g., ITO, IZO, ZITO, GZO, GIO, GITO), and conducting polymers (e.g., poly(3,4-ethylenedioxythiophene) poly(styrene-sulfonate) (PEDOT:PSS), polyaniline (PANI), polypyrrole (PPy). One or more of the composites described herein can be embodied within various organic electronic, optical, and optoelectronic devices such as organic thin film transistors (OTFTs), specifically, organic field effect transistors (OFETs), as well as sensors, capacitors, unipolar circuits, complementary circuits (e.g., inverter circuits), and the like.

Other articles of manufacture, in which graphene nanoribbons of the present invention are useful, are photovoltaics or solar cells. Compounds of the present invention can exhibit broad optical absorption and/or a very positively shifted reduction potential, making them desirable for such applications. Accordingly, the compounds described herein can be used as a n-type semiconductor in a photovoltaic design, which includes an adjacent p-type semiconductor material that forms a p-n junction. The compounds can be in the form of a thin film semiconductor, which can be deposited on a substrate to form a composite. Exploitation of compounds of the present invention in such devices is within the knowledge of a skilled artisan.

Accordingly, another aspect of the present invention relates to methods of fabricating an organic field effect transistor that incorporates a semiconductor material of the present invention. The semiconductor materials of the present invention can be used to fabricate various types of organic field effect transistors including top-gate top-contact capacitor structures, top-gate bottom-contact capacitor structures, bottom-gate top-contact capacitor structures, and bottom-gate bottom-contact capacitor structures.

In certain embodiments, OTFT devices can be fabricated with the present graphene nanoribbons on doped silicon substrates, using SiO₂ as the dielectric, in top-contact geometries. In particular embodiments, the active semiconductor layer which incorporates at least a compound of the present invention can be deposited at room temperature or at an elevated temperature. In other embodiments, the active semiconductor layer which incorporates at least a compound of the present invention can be applied by spin-coating or printing as described herein. For top-contact devices, metallic contacts can be patterned on top of the films using shadow masks, electron beam lithography and lift-off techniques, or other suitable structuring methods that are within the knowledge of a skilled artisan.

The invention is illustrated in more detail by the following examples.

### EXAMPLES

FIGS. 1 to 4 show:

FIG. 1: Polymerization and cyclodehydrogenation pathway for the surface preparation of graphene nanoribbons

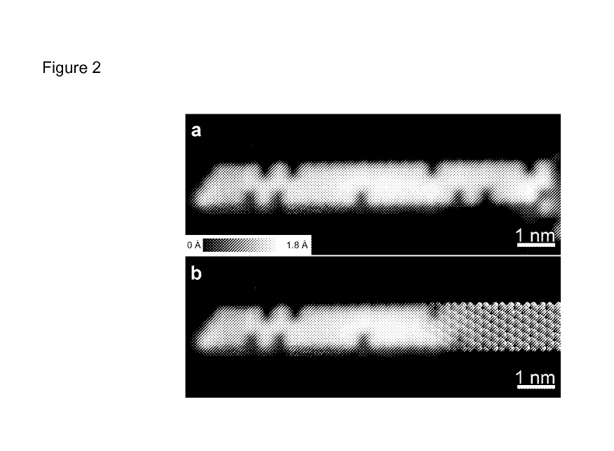

FIG. 2 (a): Scanning tunneling microscopy (STM) image (U=−0.5V, I=0.5nA, 35 K) of I-1 (m=0, n=0, R═O) after deposition and annealing on Au (111), (b) the same graphene nanoribbon overlaid with chemical model of a 9-AGNR .

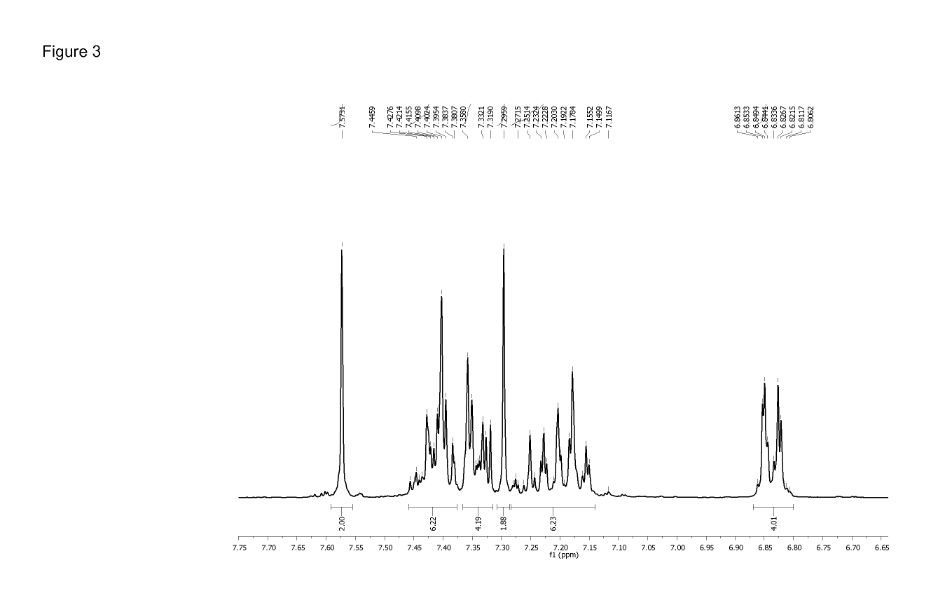

FIG. 3: ¹H NMR (300 MHz, CD₂Cl₂) of oligophenylene monomer I-1 (m=0, n=0, R═H)

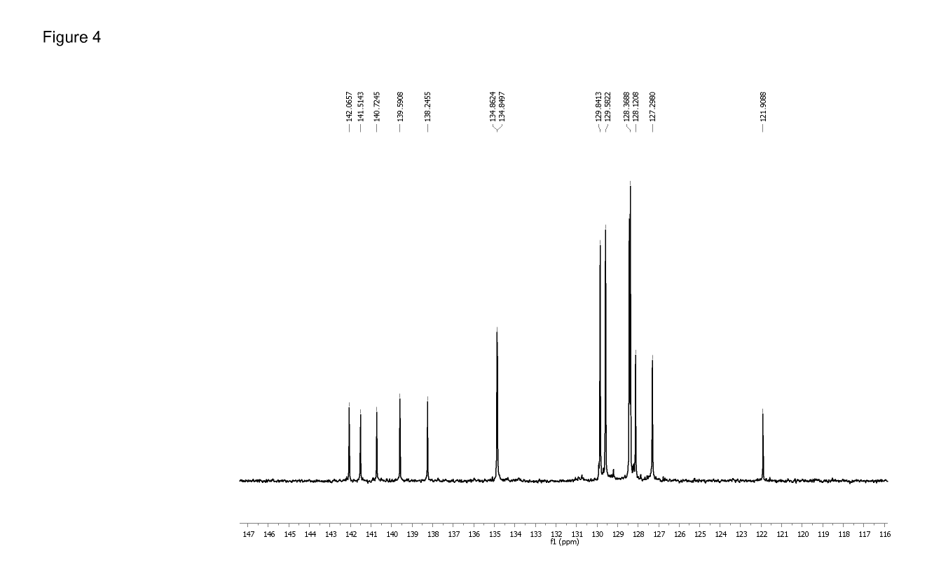

FIG. 4: ¹³C NMR (75 MHz, CD₂Cl₂) of oligophenylene monomer I-1 (m=0, n=0, R═H)

### EXAMPLE 1

### Preparation of 1,4-bis(phenyl)-2,5-bis(trimethylsilyl)benzene 3 (n=0)

20 g (60.62 mmol) 1,4-diiodobenzene 1 are dissolved in 150 ml of THF. Then, 17.05 mL (133.4 mmol, 2.2 eqv.) of trimehtylsilyl chloride are added and the reaction mixture is cooled to —78° C. Then 66.7 mL (133.4 mmol, 2.2 eqv.) of a solution of lithium diiso-propylamide in THF (2 M) are slowly added and the reaction mixture is stirred for 30 min at −78° C. The reaction mixture is carefully quenched with diluted sulfuric acid to stop the reaction. The crude product was purified by column chromatography (hexane) to yield 1,4-diiodo-2,5-bis(trimethylsilyl)-benzene as colourless crystals in 70.1%.

6.00 g (12.65 mmol) 1,4-diiodo-2,5-bis(trimethylsilyl)-benzene and 4.87 g (40.00 mmol) of phenylboronic acid 2 (n=0) were dissolved in 100.0 ml of THF. Then, 24 ml of aqueous potassium carbonate solution (2 M) and 24 mL of EtOH along with a few drops of Starks' phase transfer catalyst (Aliquat® 336 from Sigma-Aldrich) were added. After degassing by argon bubbling, 1.20 g (1.1 mmol) of tetrakis(triphenylphosphine)palladium(0) were added and the resulting mixture was heated to reflux for 24 h. After removal of the solvent, the crude product was purified by column chromatography (hexane) and recrystallized from DCM to yield 4.21 g (11.23 mmol) of 3 (n=0) as colorless crystals in 89%.

### EXAMPLE 2

### Preparation of 1,4-bis(phenyl)-2,5-dibromobenzene 4 (n=0)

4.1 g (10.94 mmol) of 3 (n=0), 5.03 g (28.226 mmol) of N-bromosuccinimide (NBS) and 2.90 g (28.226 mmol) of sodium bromide were added to 60 mL of MeOH to yield a yellowish solution. The reaction mixture was heated to reflux under inert conditions and stirred at this temperature for three days. After 1 h the reaction mixture turned turbid, after 5 h the reaction mixture turned from yellow to orange. After removal of the solvent under reduced pressure, the crude product is re-dissolved in ethyl acetate, washed with water and diluted hydrochloric acid. The final purification is carried out by column chromatography (hexane) to yield 1.49 g (3.94 mmol) of 4 (n=0) as colorless crystals in 36%.

### EXAMPLE 3

### Preparation of Arylboronic Acid Pinacol Ester 5 (n=0)

0.51 g (1.31 mmol) of 4 (n=0) are dissolved in 27 mL of anhydrous THF and cooled to −78° C. Then, 0.9 mL (1.31 mmol) of n-butyl lithium (1.6 M solution in pentane) are added dropwise. The reaction mixture turned yellowish-brown and was stirred for 2 h at this temperature. Then, 0.98 g (1.07 mL, 5.25 mmol) of (prop-2-yloxy)boronic acid pinacol ester was added and the reaction mixture was stirred overnight at room temperature. The reaction was then stopped by the addition of diluted ethanolic hydrochlorid acid. The crude product was purified by column chromatography (hexane, 6% ethyl acetate) to yield 0.13 g (0.30 mmol) of 5 (n=0) in 23%.

### EXAMPLE 4

### Preparation of Oligophenylene Monomer I-1 (m=0, n=0)

0.47 g (1.21 mmol) of 4 (n=0) and 0.11 g (0.25 mmol) of 5 (n=0) were dissolved in 20.0 ml of toluene. Then, 1 mL of aqueous potassium carbonate solution (2 M) and 5 mL of EtOH along with a few drops of Starks' phase transfer catalyst (Aliquat® 336 from Sigma-Aldrich) were added. After degassing by argon bubbling, 15 mg (0.013 mmol) of tetrakis(triphenylphosphine)palladium(0) were added and the resulting mixture was heated to reflux for 24 h. After removal of the solvent, the crude product was purified by column chromatography (hexane/ethyl acetate, gradient from 0 to 10% ethyl acetate) 99 mg (0.16 mmol) of I-1 (m=0, n=0, R=H) as yellowish crystals in 64%.

¹H NMR (300 MHz, CD₂Cl₂) δ 6.80-6.87 (m, 4H), 7.14-7.28 (m, 6H), 7.30 (s, 2H), 7.31-7.37 (m, 4H), 7.38-7.46 (m, 6H), 7.57 (s, 2H).

¹³ C NMR (75 MHz, CD₂Cl₂) δ 121.91, 127.30, 128.12, 128.37, 128.42, 129.58, 129.81, 134.85, 134.86, 138.25, 139.59, 140.72, 141.51, 142.07.

### EXAMPLE 5

### Surface Preparation of Graphene Nanoribbons

The Au(111) single crystal (Surface Preparation Laboratory, Netherlands) was used as the substrate for the growth of N=9 armchair graphene nanoribbons. First the substrate was cleaned by repeated cycles of argon ion bombardment and annealing to 470° C. and then cooled to room temperature for deposition. The precursor I-1 (m=0, n=0, R═H) monomers were deposited onto the clean surface by sublimation at rates of ˜1 Å/min. Then the Au(111) substrate was post-annealed at 200° C. for 5 min to induce polymerization and at 400° C. for 5 min to form GNRs. A variable temperature STM (VT-STM) from Omicron Nanotechnology GmbH, Germany, was used to characterize the morphology of the N=9 armchair GNR (9-AGNR) samples. FIG. 2(a) shows a high resolution STM image of a single 9-AGNR. The image was taken at U=−0.5V, I=0.5 nA, 35 K (LHe cooling). The apparent height is 1.8 angstrom, in agreement with other flat ribbon structures (J. Cai et al., Nature 2010, 466, 470.). The ribbon is about 10 nm long and 1 nm wide. There are some defects in the ribbons which are identified as missing phenyl rings which are presumably cleaved during the cyclodehydrogenation process. In FIG. 2(b), the STM image of the GNR is overlaid with a chemical model of a 9-AGNR. The agreement between model and STM image proves that 9-AGNRs can be synthesized on Au(111) surfaces from the I-1 (m=0, n=0, R=H) monomers.

1. An oligophenylene monomer of general formula I 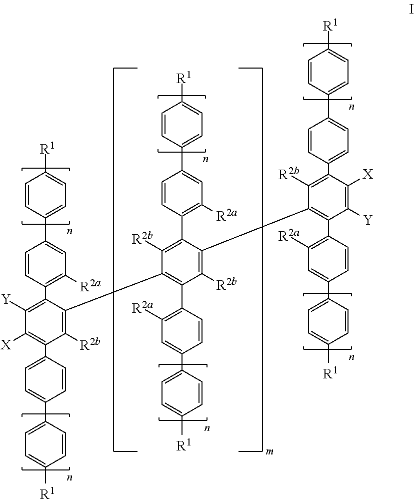
wherein
R¹ is H, halogene, —OH, —NH₂, —CN, —NO₂, or a linear or branched, saturated or unsaturated C₁-C₄₀ hydrocarbon residue, which can be substituted 1- to 5-fold with halogene (F, Cl, Br, I), —OH, —NH₂, —CN and/or —NO₂, and wherein one or more CH₂-groups can be replaced by —O—, —S—, —C(O)O—, —O—C(O)—, —C(O)—, —NH— or —NR₃—, wherein R³ is an optionally substituted C₁-C₄₀ hydrocarbon residue, or an optionally substituted aryl, alkylaryl, alkoxyaryl, alkanoyl or aroyl residue;
R^{2a} and R^{2b} are H, or optionally one or more of the pairs of adjacent R^{2a}/R^{2b} is joined to form a single bond in a six-membered carbocycle;
m is an integer of from 0 to 3;
n is 0 or 1; and
X is halogene or trifluoromethylsulfonate, and Y is H; or X is H, and Y is halogene or trifluoromethylsulfonate.

2. The oligophenylene monomer according to claim 1, wherein n is 0.

3. The oligophenylene monomer according to claim 1, wherein m is 0 or 1.

4. The oligophenylene monomer according to claim 1, wherein X is halogene or trifluoromethylsulfonate, and Y is H.

5. The oligophenylene monomer according to claim 1, wherein R^{2a} and R^{2b} are H.

6. The oligophenylene monomer according to claim 1, wherein each one of the pairs of adjacent R^{2a}/R^{2b} is joined to form a single bond in a six-membered carbocycle.

7. The oligophenylene monomer according to claim 1 of general formulae Ia, Ib, Ic or Id. 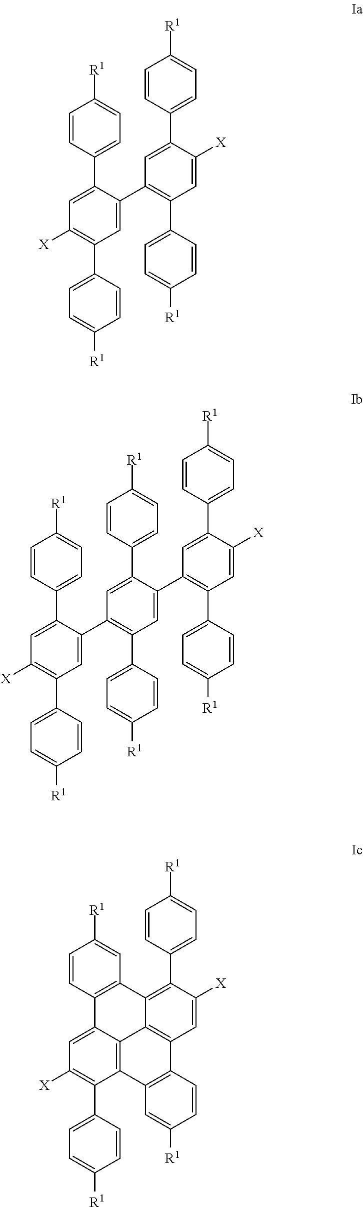 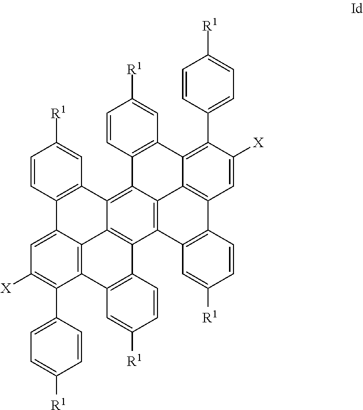
wherein
R¹ is H, halogene, —OH, —NH₂, —CN, —NO₂, or a linear or branched, saturated or unsaturated C₁-C₄₀ hydrocarbon residue, which can be substituted 1- to 5-fold with halogene (F, Cl, Br, I), —OH, —NH₂, —CN and/or —NO₂, and wherein one or more CH₂-groups can be replaced by —O—, —S—, —C(O)O—, —O—C(O)—, —C(O)—, —NH— or —NR₃—, wherein R³ is an optionally substituted C₁-C₄₀ hydrocarbon residue, or an optionally substituted aryl, alkylaryl, alkoxyaryl, alkanoyl or aroyl residue; and
X is halogene or trifluoromethylsulfonate.

8. The oligophenylene monomer according to claim 1, wherein X is H, and Y is halogene or trifluoromethylsulfonate.

9. The oligophenylene monomer according to claim 1 of general formulae Ie, If, Ig or Ih. 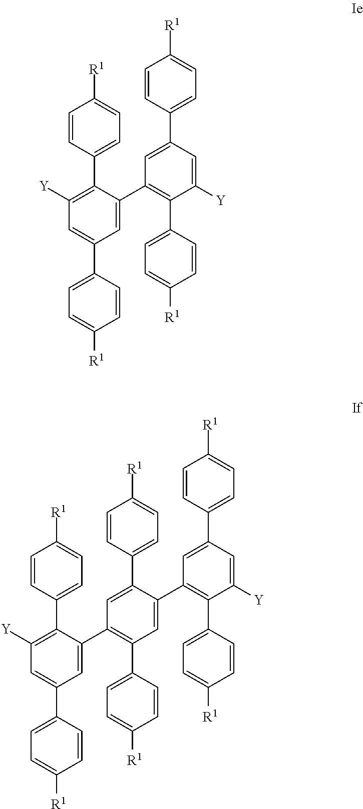 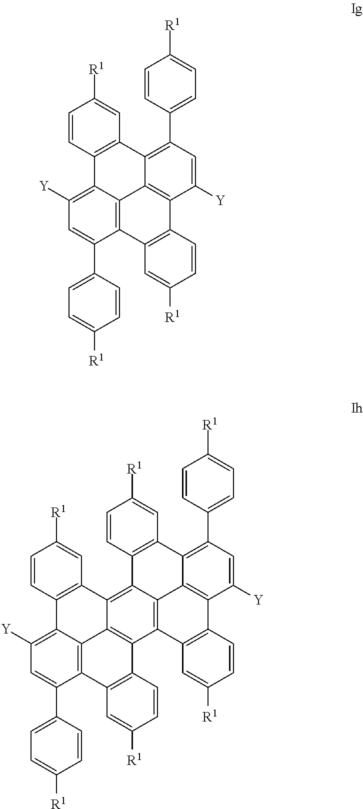
wherein
R¹ is H, halogene, —OH, —NH₂, —CN, —NO₂, or a linear or branched, saturated or unsaturated C₁-C₄₀ hydrocarbon residue, which can be substituted 1- to 5-fold with halogene (F, Cl, Br, I), —OH, —NH₂, —CN and/or —NO₂, and wherein one or more CH₂-groups can be replaced by —O—, —S—, —C(O)O—, —O—C(O)—, —C(O)—, —NH— or —NR₃—, wherein R³ is an optionally substituted C₁-C₄₀ hydrocarbon residue, or an optionally substituted aryl, alkylaryl, alkoxyaryl, alkanoyl or aroyl residue; and
Y is halogene or trifluoromethylsulfonate.

10. A polymeric precursor for the preparation of graphene nanoribbons obtained from oligophenylene monomers as defined in claim 1.

11. The polymeric precursor for the preparation of graphene nanoribbons according to claim 10, comprising at least one repeating unit selected from the group consisting of repeating units of formulae IIa, IIb, IIc, IId, IIe, IIf, IIg and IIh: 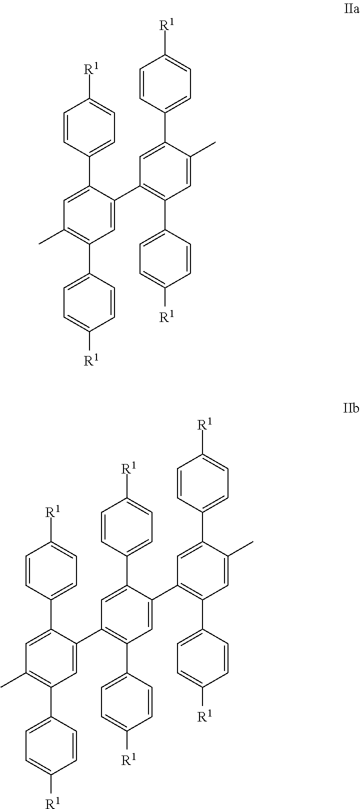 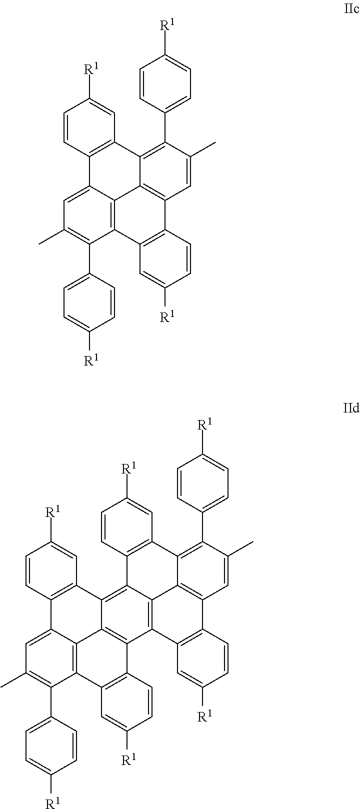 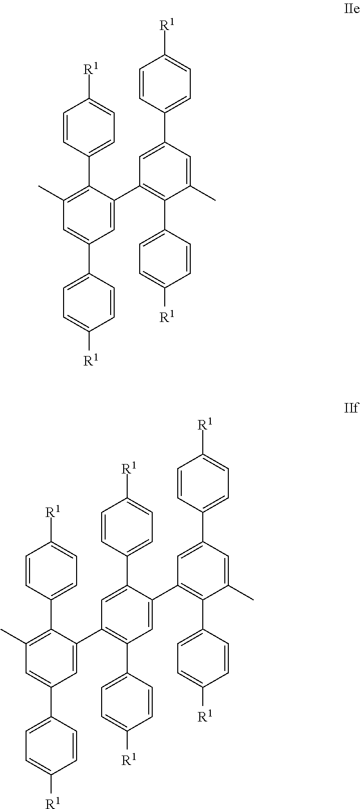 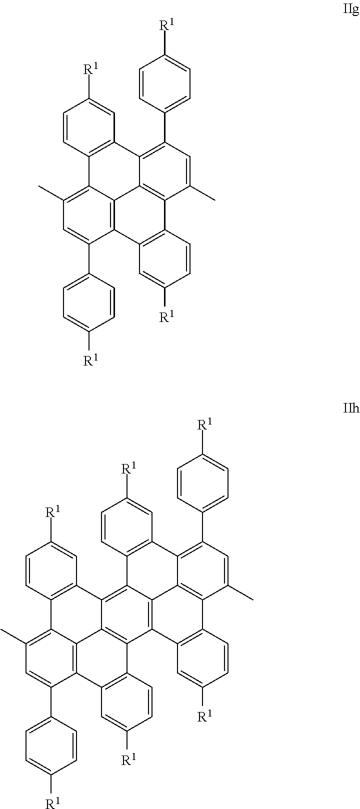
wherein
R¹ is H, halogene, —OH, —NH₂, —CN, —NO₂, or a linear or branched, saturated or unsaturated C₁—C₄₀ hydrocarbon residue, which can be substituted 1- to 5-fold with halogene (F, Cl, Br, I), —OH, —NH₂, —CN and/or —NO₂, and wherein one or more CH₂-groups can be replaced by —O—, —S—, —C(O)O—, —O—C(O)—, —C(O)—, —NH— or —NR₃—, wherein R³ is an optionally substituted C₁-C₄₀ hydrocarbon residue, or an optionally substituted aryl, alkylaryl, alkoxyaryl, alkanoyl or aroyl residue.

12. The polymeric precursor according to claim 11, obtained by polymerizing at least one oligophenylene monomer selected from the group consisting of monomers of formula Ia, Ib, Ic, Id, Ie, If, Ig and Ih: 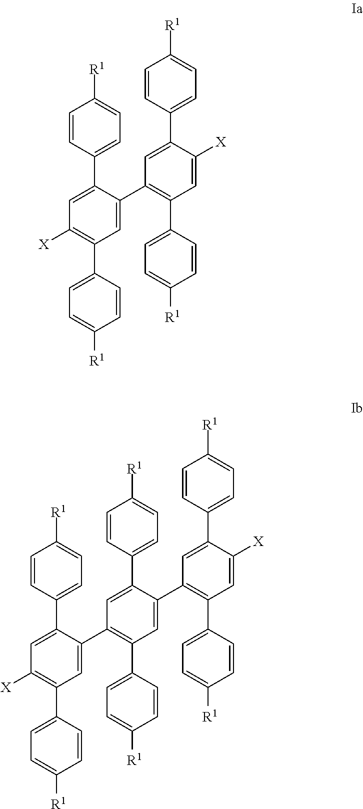 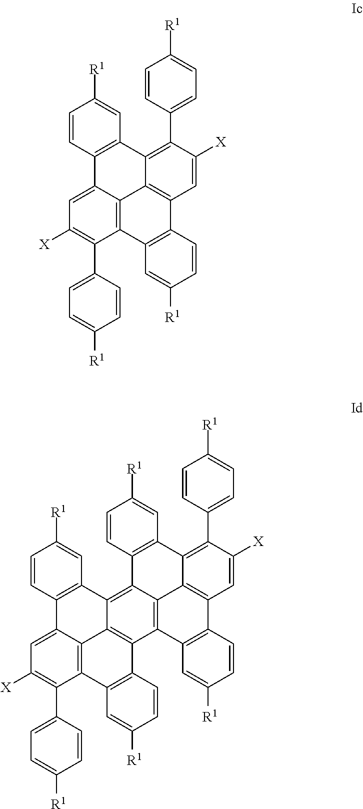 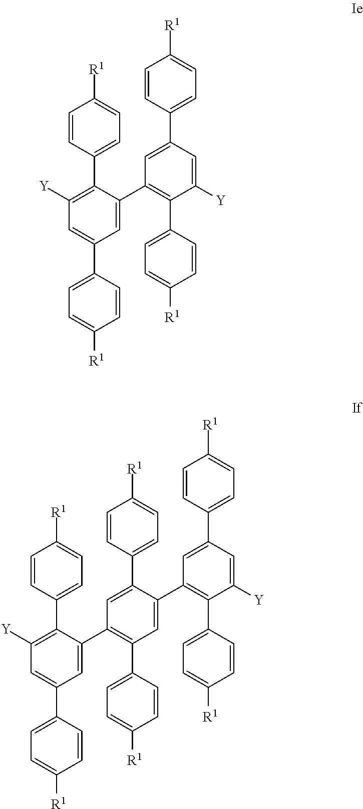 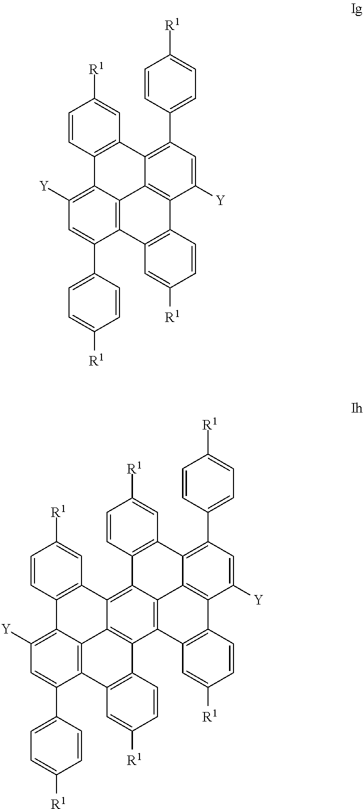
wherein
R¹ is H, halogene, —OH, —NH₂, —CN, —NO₂, or a linear or branched, saturated or unsaturated C₁-C₄₀ hydrocarbon residue, which can be substituted 1- to 5-fold with halogene (F, Cl, Br, I), —OH, —NH₂, —CN and/or —NO₂, and wherein one or more CH₂-groups can be replaced by —O—, —S—, —C(O)O—, —O—C(O)—, —C(O)—, —NH— or —NR₃—, wherein R³ is an optionally substituted C₁-C₄₀ hydrocarbon residue, or an optionally substituted aryl, alkylaryl, alkoxyaryl, alkanoyl or aroyl residue;
X is halogene or trifluoromethylsulfonate; and
Y is halogene or trifluoromethylsulfonate.

13. A graphene nonoribbon, obtained by cyclodehydrogenation of at least one polymeric precursor of claim 11.

14. The graphene nanoribbon of claim 13 prepared in a solution process.

15. The graphene nanoribbon of claim 13 prepared by direct growth of the graphene nanoribbon on a surface by polymerization of the oligophenylene monomers and cyclodehydrogenation.

16. An electronic, optical, or optoelectronic device comprising a thin film semiconductor comprising one or more graphene nanoribbons according to claim 13.

17. The device according to claim 16, wherein the device is an organic field effect transistor device, an organic photovoltaic device, or an organic light-emitting diode.

18-19. (canceled)
